SEQUENCE LISTING (5 sequences)

SEQ ID NO: 1 - protein, 6 aa, Artificial Sequence
  Synthetic
XARRAX

SEQ ID NO: 2 - DNA, 25 bp, Artificial Sequence
  Synthetic
atgcgggcgaaacttctgggaatag

SEQ ID NO: 3 - DNA, 31 bp, Artificial Sequence
  Synthetic
ttaaaatgtgtacttaagaccagcagtagtg

SEQ ID NO: 4 - DNA, 34 bp, Artificial Sequence
  Synthetic
attaatccatggcttctcgagactttatcgttta

SEQ ID NO: 5 - DNA, 34 bp, Artificial Sequence
  Synthetic
actcgggaattcttaaaagtgtacttaagaccag

(19) United States
(12) Patent Application Publication
(10) Pub. No.: US 2013/0210050 A1
(43) Pub. Date: Aug. 15, 2013

(54) PROTEASE FOR PROTEOMICS

(71) Applicants: Neil L. Kelleher, Evanston, IL (US); Cong Wu, Urbana, IL (US)

(72) Inventors: Neil L. Kelleher, Evanston, IL (US); Cong Wu, Urbana, IL (US)

(73) Assignee: Northwestern University, Evanston, IL (US)

(21) Appl. No.: 13/765,098

(22) Filed: Feb. 12, 2013

Related U.S. Application Data
(60) Provisional application No. 61/599,163, filed on Feb. 15, 2012.

(51) Int. Cl. C12Q 1/37

(52) U.S. Cl. CPC C12Q 1/37
USPC 435/23

(57) ABSTRACT

Provided herein is technology relating to proteases and proteomics and particularly, but not exclusively, to compositions comprising OmpT, methods of using OmpT, and methods of manufacturing OmpT for proteomics.

### CROSS-REFERENCE TO RELATED APPLICATIONS

The present application claims priority to pending U.S. Provisional Patent Application No. 61/599,163, filed Feb. 15, 2012.

### STATEMENT REGARDING FEDERALLY SPONSORED RESEARCH OR DEVELOPMENT

This invention was made with government support under Grant Numbers RO1 GM067193, P30 DA018310, and F30 DA026672 awarded by the National Institutes of Health, and under Grant Number DMS 0800631 awarded by the National Science Foundation. The government has certain rights in the invention.

### FIELD OF INVENTION

Provided herein is technology relating to proteases and proteomics and particularly, but not exclusively, to compositions comprising OmpT, methods of using OmpT, and methods of manufacturing OmpT for proteomics.

### BACKGROUND

Proteomics is a branch of biotechnology concerned with applying the techniques of molecular biology, biochemistry, and genetics to analyze the structure, function, and interactions of the proteins encoded by the genes of an organism. The term proteomics is somewhat analogous to the term genomics in that proteomics is the study of the proteome, the entire complement of proteins in a given biological organism at a given time, while genomics is the study of the genome, the genetic make-up of an organism. Even though the proteome of an organism is the product of that organism's genome, the proteome is larger than the genome, especially in eukaryotes, because there are more proteins than genes. This is because the genome of an organism is a rather constant entity, while the corresponding proteome differs from cell to cell and is constantly changing through its biochemical interactions with the genome and the environment. A single organism will have radically different protein expression in different parts of its body, in different stages of its life cycle, and in different environmental conditions. For example, results from the Human Genome Project indicate that there are far fewer protein coding genes in the human genome than there are proteins in the human proteome (˜22,000 genes vs. ˜400,000 proteins). The large number of proteins relative to the number of genes encoding those proteins results from mechanisms such as the alternative splicing of transcripts and the posttranslational modification of proteins. There is an increasing interest in proteomics, primarily because proteins are involved in virtually every cellular function, control every regulatory mechanism and are modified in disease (as a cause or effect).

Proteomics typically involves the analysis of the proteins contained in a biological sample, such as a cell lysate. Methods of analyzing the proteins in a biological sample are often grouped into two categories, “Bottom Up” and “Top Down” approaches, which represent two strategies for proteomic studies. Bottom Up proteomics involves enzymatic protein digestions that are optionally pre-fractionated via one- or two-dimensional separation prior to on-line reverse phase separation coupled with mass spectrometric analysis using ion trap, time-of-flight, or hybrid instruments (see, e.g., de Godoy, L. M. et al. Comprehensive mass-spectrometry-based proteome quantification of haploid versus diploid yeast. Nature 455, 1251-1254 (2008); Olsen, J. V. et al. A dual pressure linear ion trap Orbitrap instrument with very high sequencing speed. Mol Cell Proteomics 8, 2759-2769 (2009)). Top Down proteomics omits proteolysis and historically utilizes Fourier Transform Mass Spectrometry (FTMS) along with various fragmentation techniques for high-resolution tandem mass spectrometry, focusing on the complete characterization of intact proteins and their post-translational modifications (PTMs) (see, e.g., Tran, J. C. et al. Mapping intact protein isoforms in discovery mode using top-down proteomics. Nature 480, 254-258 (2011)).

While both approaches continue to mature, they each still have several intrinsic limitations (Chait, B. T. Mass spectrometry: bottom-up or top-down? Science 314, 65-66 (2006); Garcia, B. A. What does the future hold for Top Down mass spectrometry? J Am Soc Mass Spectrom 21, 193-202 (2010)). In the Bottom Up approach, tryptic peptides (typically ˜8-25 residues long; see Swaney, D. L., et al. Value of using multiple proteases for large-scale mass spectrometry-based proteomics. J Proteome Res 9, 1323-1329 (2010); Elias, J. E. & Gygi, S. P. Target-decoy search strategy for increased confidence in large-scale protein identifications by mass spectrometry. Nat Methods 4, 207-214 (2007)) are the primary unit of measurement, but their relatively small size creates three potential issues: sample complexity, the “protein inference problem” (see Nesvizhskii, A. I. & Aebersold, R. Interpretation of shotgun proteomic data: the protein inference problem. Mol Cell Proteomics 4, 1419-1440 (2005)), and the disconnection amongst combinatorial post-translational modifications. The Top Down approach handles these issues by detecting the entire protein molecule and providing information on combinations of post-translational modifications, but protein identification is less successful for proteins above 40 kDa in complex protein mixtures.

Even though significant progress has been reported for high-mass proteins (Han, X., et al. Extending top-down mass spectrometry to proteins with masses greater than 200 kilodaltons. Science 314, 109-112 (2006)), a robust measurement platform based on characterizing 2-20 kDa peptides could marry the positive aspects of both Bottom Up and Top Down proteomics. Such a platform, coupled with electron-based fragmentation methods (e.g., electron capture/transfer dissociation) (Syka, J. E., et al. Peptide and protein sequence analysis by electron transfer dissociation mass spectrometry. Proc Natl Acad Sci USA 101, 9528⁻9533 (2004); Taouatas, N., et al. Straightforward ladder sequencing of peptides using a Lys-N metalloendopeptidase. Nat Methods 5, 405-407 (2008)), would exploit the favorable kinetics of electron capture/transfer for highly charged peptide ions while still achieving precise localization of even-labile post-translational modifications on a chromatographic time scale (Taouatas, N. et al. Strong cation exchange-based fractionation of Lys-N-generated peptides facilitates the targeted analysis of post-translational modifications. Mol Cell Proteomics 8, 190-200 (2009)).

Previously, conventional proteomics approaches for interrogating high-mass proteins identified two technologies that could provide a foundation for “Middle Down” proteomics: 1) a size-dependent protein fractionation technique; and 2) a robust but restricted proteolysis method (see, e.g., Forbes, A. J., et al. Toward efficient analysis of >70 kDa proteins with 100% sequence coverage. Proteomics 1, 927-933 (2001)). The first feature is provided in some technologies by a continuous tube-gel electrophoresis technique that has achieved the size-dependent fractionation of a complex proteome with high recoveries of proteins in liquid phase (Lee, J. E. et al. A robust two-dimensional separation for top-down tandem mass spectrometry of the low-mass proteome. J Am Soc Mass Spectrom 20, 2183-2191 (2009); Tran, J. C. & Doucette, A. A. Multiplexed size separation of intact proteins in solution phase for mass spectrometry. Anal Chem 81, 6201-6209 (2009); Tran, J. C. & Doucette, A. A. Gel-eluted liquid fraction entrapment electrophoresis: an electrophoretic method for broad molecular weight range proteome separation. Anal Chem 80, 1568-1573 (2008)).

In addition, initial efforts to develop the second technology utilized restricted proteolysis with the proteases Glu-C, Lys-C, or Asp-N to produce larger peptides and preserve multiple PTMs for the targeted proteomics (Garcia, B. A., et al. Pervasive combinatorial modification of histone H3 in human cells. Nat Methods 4, 487-489 (2007); Jiang, L. et al. Global assessment of combinatorial post-translational modification of core histones in yeast using contemporary mass spectrometry. LYS4 trimethylation correlates with degree of acetylation on the same H3 tail. J Biol Chem 282, 27923-27934 (2007); Phanstiel, D. et al. Mass spectrometry identifies and quantifies 74 unique histone H4 isoforms in differentiating human embryonic stem cells. Proc Natl Acad Sci USA 105, 4093-4098 (2008); Siuti, N. & Kelleher, N. L. Efficient readout of posttranslational codes on the 50-residue tail of histone H3 by high-resolution MS/MS. Anal Biochem 396, 180-187 (2010); Wu, S. L., et al. Extended Range Proteomic Analysis (ERPA): a new and sensitive LC-MS platform for high sequence coverage of complex proteins with extensive post-translational modifications-comprehensive analysis of beta-casein and epidermal growth factor receptor (EGFR). J Proteome Res 4, 1155-1170 (2005)), while later efforts on the proteome-scale have employed Lys-C or Lys-N digestions (Boyne, M. T. et al. Tandem mass spectrometry with ultrahigh mass accuracy clarifies peptide identification by database retrieval. J Proteome Res 8, 374-379 (2009); Scholten, A. et al. In-depth quantitative cardiac proteomics combining electron transfer dissociation and the metalloendopeptidase Lys-N with the SILAC mouse. Mol Cell Proteomics 10, 0111 008474 (2011)). Nonetheless, these enzymes produce peptides only marginally longer than tryptic peptides in large-scale proteomic studies, offering limited improvement in peptide size. Furthermore, microwave-assisted acid hydrolysis techniques generated peptides in the 3-10 kDa range with selective cleavage at aspartic acid residues. This approach improved ribosomal proteome coverage, but the peptides produced were still relatively small (average: 3.2 kDa) (see Hauser, N. J., et al. Electron transfer dissociation of peptides generated by microwave D-cleavage digestion of proteins. J Proteome Res 7, 1867-1872 (2008); Cannon, J. et al. High-throughput middle-down analysis using an orbitrap. J Proteome Res 9, 3886-3890 (2010); Swatkoski, S. et al. Evaluation of microwave-accelerated residue-specific acid cleavage for proteomic applications. J Proteome Res 7, 579-586 (2008); Hauser, N. J. & Basile, F. Online microwave D-cleavage LC-ESI-MS/MS of intact proteins: site-specific cleavages at aspartic acid residues and disulfide bonds. J Proteome Res 7, 1012-1026 (2008)).

### SUMMARY

Provided herein is technology related to the protease OmpT to achieve a robust, yet restricted, proteolysis of complex mixtures of polypeptides. OmpT cleaves polypeptides between dibasic amino acid residues (e.g., K/R-K/R) (Dekker, N., et al. Substrate specificity of the integral membrane protease OmpT determined by spatially addressed peptide libraries. Biochemistry 40, 1694-1701 (2001); McCarter, J. D. et al. Substrate specificity of the Escherichia coli outer membrane protease OmpT. J Bacteriol 186, 5919-5925 (2004); Keijiro Sugimura, T. N. Purification, Characterization, and Primary Structure of Escherichia coli Protease VII with Specificity for Paired Basic Residues: Identity of Protease VII and OmpT. Journal of Bacteriology 170, 5625-5632 (1988); Sugimura, K. & Higashi, N. A novel outer-membrane-associated protease in Escherichia coli. J Bacteriol 170, 3650-3654 (1988)), instead of after single K or R residues as in the case for trypsin. OmpT has a substrate-dependent k_{cat}/Kₘ of 10⁴-10⁸ s⁻¹M⁻¹ in vitro for a wide diversity of substrates (McCarter, J. D. et al. Substrate specificity of the Escherichia coli outer membrane protease OmpT. J Bacteriol 186, 5919-5925 (2004); Varadarajan, N., et al. Highly active and selective endopeptidases with programmed substrate specificities. Nat Chem Biol 4, 290-294 (2008); Kramer, R. A., et al. In vitro folding, purification and characterization of Escherichia coli outer membrane protease ompT. Eur J Biochem 267, 885-893 (2000); Olsen, M. J. et al. Function-based isolation of novel enzymes from a large library. Nat Biotechnol 18, 1071-1074 (2000); Varadarajan, N., et al. Engineering of protease variants exhibiting high catalytic activity and exquisite substrate selectivity. Proc Natl Acad Sci USA 102, 6855-6860 (2005); Vandeputte-Rutten, L. et al. Crystal structure of the outer membrane protease OmpT from Escherichia coli suggests a novel catalytic site. EMBO J 20, 5033-5039 (2001); Okuno, K., et al. An analysis of target preferences of Escherichia coli outer-membrane endoprotease OmpT for use in therapeutic peptide production: efficient cleavage of substrates with basic amino acids at the P4 and P6 positions. Biotechnol Appl Biochem 36, 77-84 (2002)). For reference, trypsin has a k_{cat}/Kₘ between 10⁶-10⁷s⁻¹M⁻¹ (Hedstrom, L., et al. Converting trypsin to chymotrypsin: the role of surface loops. Science 255, 1249-1253 (1992); Graf, L. et al. Electrostatic complementarity within the substrate-binding pocket of trypsin. Proc Natl Acad Sci USA 85, 4961-4965 (1988); Corey, D. R., et al. Trypsin specificity increased through substrate-assisted catalysis. Biochemistry 34, 11521-11527 (1995)).

In contrast to chemical methods, such as cyanogen bromide (CNBr, which cleaves after methionine and chemically modifies the protein) (Erhard, G. in Methods in Enzymology Vol. Volume 11 (ed. C. H. W. Hirs) 238-255 (Academic Press, 1967); Witkop, B. Nonenzymatic methods for the preferential and selective cleavage and modification of proteins. Adv Protein Chem 16, 221-321 (1961)) or BNPS-skatole (which cleaves after tryptophan) (Hunziker, P. E., et al. Peptide fragmentation suitable for solid-phase microsequencing. Use of N-bromosuccinimide and BNPS-skatole (3-bromo-3-methyl-2-[(2-nitrophenyl)thio]-3H-indole). Biochem J 187, 515-519 (1980); Rahali, V. & Gueguen, J. Chemical cleavage of bovine beta-lactoglobulin by BNPS-skatole for preparative purposes: comparative study of hydrolytic procedures and peptide characterization. J Protein Chem 18, 1-12 (1999)), the present technology has improved specificity and robustness, and is associated with minimal side reactions.

OmpT is derived from Escherichia coli (e.g., Escherichia coli K12) and belongs to the omptin protease family together with four other members (Mangel, W. F. et al. Omptin: an Escherichia coli outer membrane proteinase that activates plasminogen. Methods Enzymol 244, 384-399 (1994)). While its function in vivo has not been fully characterized, OmpT is implicated in the degradation of many recombinantly expressed proteins in E. coli (Pritchard, A. E., et al. In vivo assembly of the tau-complex of the DNA polymerase III holoenzyme expressed from a five-gene artificial operon. Cleavage of the tau-complex to form a mixed gamma-tau-complex by the OmpT protease. J Biol Chem 271, 10291-10298 (1996); Grodberg, J. & Dunn, J. J. ompT encodes the Escherichia coli outer membrane protease that cleaves T7 RNA polymerase during purification. J Bacteriol 170, 1245-1253 (1988); White, C. B., et al. A novel activity of OmpT. Proteolysis under extreme denaturing conditions. J Biol Chem 270, 12990-12994 (1995)). As provided herein, embodiments of the present technology provide compositions comprising OmpT and methods using OmpT as a reagent to generate peptides having a mass of greater than 2 kDa for Middle Down proteomics.

Accordingly, provided herein is technology related to a method for identifying a polypeptide, the method comprising contacting the polypeptide with an OmpT protease to produce a fragment and analyzing the fragment by mass spectrometry to generate a mass spectrum. The technology is not limited in the type or source of the protease that is used to contact and digest the polypeptide. For example, in some embodiments the OmpT protease is a recombinant OmpT protease. In some embodiments, the OmpT protease is cloned from Escherichia coil. In some embodiments, the OmpT protease is an endogenous protease isolated from Escherichia coil. In some embodiments, the OmpT protease is a mutant OmpT protease; for example, in some embodiments the OmpT protease has amino acid substitutions at one or more positions.

The technology relates to digesting a polypeptide, for example, to provide a sample for mass spectrometry. It is contemplated that the technology is useful for any other test, analysis, or method in which a polypeptide digest is appropriate. In some embodiments, the method produces a fragment that has a mass that is greater than 2 kDa. In some embodiments, the method produces a fragment that has a mass that is greater than 10 kDa. In some embodiments, the method produces a fragment that has a mass that is greater than 30 kDa.

Some embodiments provide a solution comprising the OmpT protease and a polypeptide. In some embodiments, the composition of the solution is controlled to provide a particular milieu in which to digest the polypeptide. For example, in some embodiments the polypeptide is contacted with a denaturant to expose (e.g., denature or linearize) the polypeptide for contacting with the OmpT protease. Accordingly, embodiments of the technology provided herein provide methods in which contacting the polypeptide with the OmpT protease occurs in the presence of a denaturant. Particular embodiments provide, for example, that the contacting occurs in approximately 2-3 M urea, at approximately 22° C., and at about a pH of 6. Moreover, in some embodiments the temperature and the amounts of the OmpT protease and the polypeptide are controlled; for example, some embodiments provide a method in which the contacting occurs for approximately 10-16 hours or overnight and a ratio of the polypeptide to the OmpT protease that is approximately 25:1 to 75:1.

The technology described herein finds use in identifying a polypeptide by mass spectrometry. Accordingly, analyzing the fragment produced by OmpT digestion produces data, e.g., a mass spectrum, that provides information about the fragment and the polypeptide (e.g., its identity, origin, sequence, post-translational modifications, etc.). In some embodiments, the data, e.g., the mass spectrum, is compared to a database to obtain the information about the fragment and the polypeptide. In some embodiments, the data, e.g., the mass spectrum, is used to differentiate between different isoforms of the polypeptide. That is, in some embodiments, the fragment has a mass that is different than a second mass of a second fragment, wherein the second fragment results from contacting another isoform of the polypeptide with an OmpT protease.

In some embodiments, a sample is fractionated and/or purified to provide the polypeptide. For example, in some embodiments of the technology, the method provides the polypeptide separated based on molecular weight by continuous tube-gel electrophoresis. In some embodiments, a proteome is fractionated or purified, e.g., to provide a fraction comprising more than one polypeptide.

The methods provided herein relate to a method for identifying a polypeptide, the method comprising contacting the polypeptide with a protease that specifically cleaves at a two-amino acid recognition site to produce a fragment and analyzing the fragment by mass spectrometry to generate a mass spectrum. In some embodiments, the two-amino acid recognition site comprises a dibasic site. In some embodiments the first amino acid of the two-amino acid recognition site is a lysine or an arginine and the second amino acid of the two-amino acid recognition site is a lysine or an arginine. In some embodiments, the first amino acid of the two-amino acid recognition site is a lysine or an arginine and the second amino acid of the two-amino acid recognition site is an alanine. In some embodiments, the first amino acid of the two-amino acid recognition site is a lysine or an arginine and the second amino acid of the two-amino acid recognition site is a lysine, an arginine, an alanine, a serine, a glycine, a valine, an isoleucine, or a leucine.

In certain embodiments, the present invention provides altered forms of OmpT wherein its amino acid sequence does not comprise dibasic amino acid residues. Such altered forms of OmpT (e.g., wherein its amino acid sequence does not comprise dibasic amino acid residues) retains the ability to cleave polypeptides between dibasic amino acid residues (e.g., K/R-K/R). In some embodiments, such altered forms of OmpT (e.g., wherein its amino acid sequence does not comprise dibasic amino acid residues) are generated through replacing its dibasic amino acid residues with at least one non-basic amino acid residue.

In certain embodiments, the present invention provides altered forms of OmpT where it is able to function (e.g., able to cleave polypeptides between dibasic amino acid residues (e.g., K/R-K/R)) in any urea concentration. For example, the present invention provides altered forms of OmpT able to function (e.g., able to cleave polypeptides between dibasic amino acid residues (e.g., K/R-K/R)) at urea concentrations at or above 3M.

Further provided by the technology are embodiments of kits, for example, a kit comprising an OmpT protease, a buffer, a negative control polypeptide, and/or a positive control polypeptide. Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a plot showing in silico digestions of the human proteome with various proteolytic methods. A human database with 88,506 sequences was plotted directly (intact) or digested in silico by enzymatic methods (trypsin, OmpT, Lys-C, Glu-C, Asp-N, Arg-C) or a chemical method (CNBr) with no missed cleavages. OmpT was assumed to cleave RR, KR, RK, and KK sites. The numbers of generated peptides were plotted versus mass bins from 1 kDa to 30 kDa on a semi-logarithmic scale.

FIG. 2a shows the major species (peptides 1-3) in a base peak mass spectrum from a nanoLC-MS/MS analysis. FIG. 2b shows the charge state distributions of peptides 1-3. FIG. 2c shows the tandem mass spectra of particular charge states from FIG. 2b. The masses of identified peptides and their raw p scores are shown. FIG. 2d shows an alignment of identified OmpT peptides with a schematic of the original GAPDH sequence at the top. Peptide cleavage sites are illustrated and N and C represent the protein N- and C-termini.

FIG. 3 shows a representation of a typical nanoLC-MS/MS analysis of a secondary continuous tube-gel electrophoresis fraction. FIG. 3a shows the base peak mass spectrum. FIG. 3b shows intact change state distributions for three selected OmpT peptide species having the indicated monoisotopic masses. FIG. 3c shows fragmentation spectra of the three corresponding precursors from FIG. 3b. Also shown are the identified proteins from which these OmpT peptides were derived and their q values.

FIG. 4 shows drawings depicting examples of identifying a specific protein isoform based on proteotypic OmpT peptides. Cleavage sites are shown for each identified OmpT peptide. The different sequence regions in isoform alignments are marked between dashed lines. Peptides covering the distinct part of a certain isoform are shaded in black; peptides covering the common regions of all isoforms are in grey. FIG. 4a shows a drawing of peptides 1 and 2 (10.8 kDa and 5.4 kDa, respectively) from a proteotypic sequence region of one L-lactate dehydrogenase A chain isoform 1; the identified OmpT peptides cover the entire isoform-1 sequence. FIG. 4b depicts how peptide 3 (9.8 kDa) leads to the specific identification of isoform A1-A of heterogeneous nuclear ribonucleoprotein A1. The sequence coverage of this isoform is 98%. In the drawing of FIG. 4c, among the identified peptides from heat shock protein 90-beta (shaded in grey), peptide 4 (8.9 kDa) harbors up to two phosphorylation modifications. FIG. 4d shows peptide 5 (7.2 kDa), which was identified with an N-terminal acetylation and an N6-(4-amino-2-hydroxybutyl)-lysine from eukaryotic translation initiation factor 5A-1. FIG. 4e shows a peptide (peptide 6, 3.8 kDa) that contains two dimethylarginines from the C-terminus of 40S ribosomal protein S10.

FIG. 5 shows peptide histograms and a schematic of the protease recognition site. FIG. 5a shows the mass distribution of identified OmpT peptides in comparison with tryptic peptides. FIG. 5b shows a histogram of proteins identified with OmpT peptides from the HeLa proteome. FIG. 5c shows the consensus of OmpT recognition sequences from the P4 through the P4′ sites. Data shown are for OmpT peptides below ˜15 kDa.

### DETAILED DESCRIPTION

OmpT is a rare-cutting protease for Middle Down proteomics. The larger-sized OmpT peptides improve sequence coverage, isoform-specific protein identifications, and the chance of characterizing PTM combinations. Additionally, OmpT is resistant to both denaturants and surfactants, allowing extensive denaturation of the three-dimensional structure of large substrate proteins for robust proteolysis in strongly solubilizing conditions.

Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The terms “protein” and “polypeptide” and “peptide” refer to compounds comprising amino acids joined via peptide bonds. A “protein” or “polypeptide” encoded by a gene is not limited to the amino acid sequence encoded by the gene, but includes any modifications (e.g., post-translational modifications) of the protein or its constituent amino acids.

As used herein, the term, “synthetic polypeptide,” “synthetic peptide”, and “synthetic protein” refer to peptides, polypeptides, and proteins that are produced by a recombinant process (i.e., expression of exogenous nucleic acid encoding the peptide, polypeptide, or protein in an organism, host cell, or cell-free system) or by chemical synthesis.

As used herein, the term “protein of interest” refers to a protein encoded by a nucleic acid of interest.

As used herein, the term “proteolysis” is the biochemical process of breaking peptide bonds between amino acids in a protein. This process is carried out by enzymes called peptidases, proteases, or proteolytic cleavage enzymes. The nomenclature of cleavage site positions within a substrate polypeptide are described, e.g., in Schechter, I. & Berger, A. On the active site of proteases. 3. Mapping the active site of papain; specific peptide inhibitors of papain. Biochem Biophys Res Commun, 32, 898-902 (1968); Schechter, I. & Berger, A. On the size of the active site in proteases. I. Papain. Biochem Biophys Res Commun, 27, 157-162 (1967). The cleavage site is designated as being the peptide bond between the “P1” and “P1” amino acids, divergently incrementing the numbering in the N- and C-terminal directions from the cleaved peptide bond. P1, P2, P3, P4, etc. are used on the N-terminal side and P1′, P2′, P3′, P4′, etc. are used on the C-terminal side of the cleavage site.

As used herein, the term “native” (or wild type) when used in reference to a protein refers to proteins encoded by the genome of a cell, tissue, or organism, other than one manipulated to produce synthetic proteins.

Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “nascent” when used in reference to a protein refers to a newly synthesized protein, which has not been subject to post-translational modifications, which includes but is not limited to glycosylation and polypeptide shortening. The term “mature” when used in reference to a protein refers to a protein which has been subject to post-translational processing and/or which is in a cellular location (such as within a membrane or a multi-molecular complex) from which it can perform a particular function which it could not if it were not in the location. Mature proteins may also refer to proteins after post-translational processing, such as enzyme cleavage to convert a protein (e.g., a pre-enzyme) into an active protein (e.g., a mature enzyme). Therefore, the sequence of a “nascent protein” and a “mature protein” can be different.

The term “portion” when used in reference to a protein (as in “a portion of a given protein”) refers to fragments of that protein. The fragments may range in size from two amino acid residues to the entire amino sequence minus one amino acid (for example, the range in size includes 4, 5, 6, 7, 8, 9, 10, or more amino acids up to the entire amino acid sequence minus one amino acid).

The term “homolog” or “homologous” when used in reference to a polypeptide refers to a high degree of sequence identity between two polypeptides, or to a high degree of similarity between the three-dimensional structure, or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned (e.g., by a sofware program (e.g., GAP or BESTFIT)) using default gap weights), share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The term “domain” when used in reference to a polypeptide refers to a subsection of the polypeptide that possesses a unique structural and/or functional characteristic; typically, this characteristic is similar across diverse polypeptides. The subsection typically comprises contiguous amino acids, although it may also comprise amino acids that act in concert or that are in close proximity due to folding or other configurations. Examples of a protein domain include transmembrane domains and glycosylation sites. For example, domains include those portions of a polypeptide chain that can form an independently folded structure within a protein made up of one or more structural motifs and/or that is recognized by virtue of a functional activity, such as proteolytic activity. Generally, domains are responsible for discrete functional properties of proteins, and in many cases may be added, removed or transferred to other proteins without loss of function of the remainder of the protein and/or of the domain.

A protein can have one, or more than one, distinct domains. For example, a domain can be identified, defined or distinguished by homology of the sequence therein to related family members, such as homology to motifs that define a protease domain or a gla domain. In another example, a domain can be distinguished by its function, such as by proteolytic activity, or an ability to interact with a biomolecule, such as DNA binding, ligand binding, and dimerization. A domain independently can exhibit a biological function or activity such that the domain independently or fused to another molecule can perform an activity, such as, for example proteolytic activity or ligand binding. A domain can be a linear sequence of amino acids or a non-linear sequence of amino acids. Many polypeptides contain a plurality of domains. Some domains are known and can be identified by those of skill in the art. It is to be understood that it is well within the skill in the art to recognize particular domains by name. If needed, appropriate software can be employed to identify domains.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “nucleotide sequence of interest” or “nucleic acid sequence of interest” refers to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The term “structural” when used in reference to a gene or to a nucleotide or nucleic acid sequence refers to a gene or a nucleotide or nucleic acid sequence whose ultimate expression product is a protein (such as an enzyme or a structural protein), an rRNA, an sRNA, a tRNA, etc.

The term “wild-type” when made in reference to a gene refers to a gene that has the characteristics of a gene isolated from a naturally occurring source. The term “wild-type” when made in reference to a gene product (e.g., a polypeptide) refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The nomenclature used to describe variants of nucleic acids or proteins specifies the type of mutation and base or amino acid changes. For a nucleotide substitution (e.g., 76A>T), the number is the position of the nucleotide from the 5′ end, the first letter represents the wild type nucleotide, and the second letter represents the nucleotide which replaced the wild type. In the given example, the adenine at the 76th position was replaced by a thymine. If it becomes necessary to differentiate between mutations in genomic DNA, mitochondrial DNA, complementary DNA (cDNA), and RNA, a simple convention is used. For example, if the 100th base of a nucleotide sequence is mutated from G to C, then it would be written as g.100G>C if the mutation occurred in genomic DNA, m.100G>C if the mutation occurred in mitochondrial DNA, c.100G>C if the mutation occurred in cDNA, or r.100g>c if the mutation occurred in RNA.

For amino acid substitution (e.g., D111E), the first letter is the one letter code of the wild type amino acid, the number is the position of the amino acid from the N-terminus, and the second letter is the one letter code of the amino acid present in the mutation. Nonsense mutations are represented with an X for the second amino acid (e.g. D111X). For amino acid deletions (e.g. ΔF508, F508del), the Greek letter Δ (delta) or the letters “del” indicate a deletion. The letter refers to the amino acid present in the wild type and the number is the position from the N terminus of the amino acid where it is present in the wild type. Intronic mutations are designated by the intron number or cDNA position and provide either a positive number starting from the G of the GT splice donor site or a negative number starting from the G of the AG splice acceptor site. g.3′+7G>C denotes the G to C substitution at nt +7 at the genomic DNA level. When the full-length genomic sequence is known, the mutation is best designated by the nucleotide number of the genomic reference sequence. See den Dunnen & Antonarakis, “Mutation nomenclature extensions and suggestions to describe complex mutations: a discussion”. Human Mutation 15: 7-12 (2000); Ogino S, et al., “Standard Mutation Nomenclature in Molecular Diagnostics: Practical and Educational Challenges”, J. Mol. Diagn. 9(1): 1-6 (February 2007).

As used herein, the one-letter codes for amino acids refer to standard IUB nomenclature as described in “IUPAC-IUB Nomenclature of Amino Acids and Peptides” published in Biochem. J, 1984, 219, 345-373; Eur. J Biochem., 1984, 138, 9-37; 1985, 152, 1; Internat. J Pept. Prot. Res., 1984, 24, following p 84; J Biol. Chem., 1985, 260, 14-42; Pure Appl. Chem., 1984, 56, 595-624; Amino Acids and Peptides, 1985, 16, 387-410; and in Biochemical Nomenclature and Related Documents, 2nd edition, Portland Press, 1992, pp 39-67.

As used herein, the term “isoform” (also known as an “isozyme” if the protein is an enzyme) refers to proteins and/or enzymes with same or similar function but that differ in amino acid and/or nucleotide sequences. Isoforms exist by multiple mechanisms, such as different gene loci, multiple alleles (also called allelomorphs, allelozymes, or allozymes), different subunit interaction, different splice forms, or different post-translational modification, and can usually be separated by electrophoresis or some other separation technique known in the art.

The term “polymorphic locus” refers to a genetic locus present in a population that shows variation between members of the population (i.e., the most common allele has a frequency of less than 0.95). Thus, “polymorphism” refers to the existence of a character in two or more variant forms in a population. A “single nucleotide polymorphism” (or SNP) refers a genetic locus of a single base which may be occupied by one of at least two different nucleotides. In contrast, a “monomorphic locus” refers to a genetic locus at which little or no variations are seen between members of the population (generally taken to be a locus at which the most common allele exceeds a frequency of 0.95 in the gene pool of the population).

A “frameshift mutation” refers to a mutation in a nucleotide sequence, usually resulting from insertion or deletion of a single nucleotide (or two or four nucleotides) which results in a change in the correct reading frame of a structural DNA sequence encoding a protein. The altered reading frame usually results in the translated amino-acid sequence being changed or truncated.

A “splice mutation” refers to any mutation that affects gene expression by affecting correct RNA splicing. Splicing mutation may be due to mutations at intron-exon boundaries which alter splice sites.

The term “detection assay” refers to an assay for detecting the presence or absence of a wild-type or variant nucleic acid sequence (e.g., mutation or polymorphism) in a given allele of a particular gene, or for detecting the presence or absence of a particular protein or the activity or effect of a particular protein or for detecting the presence or absence of a variant of a particular protein.

The term “sample” is used in its broadest sense. In one sense it can refer to an animal cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

Embodiments of the Technology

Mass spectrometry

In some embodiments, determining the mass of target fragments employs mass spectrometry. Mass spectrometry (MS) is an analytical technique that measures the mass-to-charge ratio of charged particles and ions. MS methods filter, detect, and measure ions based on their mass-to-charge (e.g., “m/z”) ratio. It is often used for characterizing the chemical structures of polypeptides (e.g., proteins and small peptides). In an MS experiment, a chemical compound is ionized to generate charged molecules or molecule fragments and then their mass-to-charge ratios are measured. For example, in a typical MS method a sample is vaporized and its components are ionized (e.g., by impacting them with an electron beam), which results in the formation of charged particles (ions). Then, the ions are separated according to their mass-to-charge ratio by an electromagnetic field and the ions are detected. Data are typically presented as a mass spectrum.

The technique has both qualitative and quantitative applications. For example, MS is used to identify unknown compounds (e.g., a polypeptide), to determine the isotopic composition of elements in a molecule, and to determine the structure of a compound by observing its fragmentation. Other uses include quantifying the amount of a compound in a sample.

Accordingly, mass spectrometry is an emerging method for the characterization and sequencing of proteins and proteomes. Two approaches are used for characterizing proteins. In the first, intact proteins are ionized and then introduced to a mass analyzer. This approach is referred to as “top-down” strategy of protein analysis. In the second, proteins are enzymatically digested into smaller peptides using proteases such as trypsin or pepsin, either in solution or in a gel after electrophoretic separation. Other proteolytic agents are also used. The collection of peptide products is then introduced into the mass analyzer. The characteristic pattern of peptides can be used to identify the protein (“peptide mass fingerprinting” or “PMF”). These procedures of protein analysis are also referred to as the “bottom-up” approach.

For a MS analysis in general, one or more molecules of interest are ionized and the ions are subsequently introduced into a mass spectrometer where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces”; U.S. Pat. No. 6,107,623, entitled “Methods and Apparatus for Tandem Mass Spectrometry”; U.S. Pat. No. 6,268,144, entitled “DNA Diagnostics Based On Mass Spectrometry”; U.S. Pat. No. 6,124,137, entitled “Surface-Enhanced Photolabile Attachment And Release For Desorption And Detection Of Analytes”; Wright et al., Prostate Cancer and Prostatic Diseases 2: 264-76 (1999); and Merchant and Weinberger, Electrophoresis 21: 1164-67 (2000), each of which is hereby incorporated by reference in its entirety and for all purposes, including all tables, figures, and claims. The terms “integrated intensity,” “mass spectral integrated area,” “integrated mass spectral intensity,” and the like refer to the area under a mass spectrometric curve corresponding to the amount of a molecular ion having a particular main isotope m/z, as is well known in the art.

Different types of MS apparatuses are employed for MS analysis. For example, in a “quadrupole” or “quadrupole ion trap” instrument, ions in an oscillating radio frequency field experience a force proportional to the DC potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

Moreover, one can often acquire additional useful information by employing “tandem mass spectrometry”, also designated by the term “MS/MS.” In this technique, a first, or parent, ion generated from a molecule of interest is filtered in an MS instrument and these parent ions are subsequently fragmented to yield one or more second, or daughter, ions that are then analyzed in a second MS procedure. By careful selection of parent ions, only ions produced by certain analytes are passed to the fragmentation chamber, where collision with atoms of an inert gas produces the daughter ions. Because both the parent and daughter ions are produced in a reproducible fashion under a given set of ionization and fragmentation conditions, the MS/MS technique can provide an extremely powerful analytical tool. For example, the combination of filtration and fragmentation is used to eliminate interfering substances and is particularly useful for complex samples such as biological samples. Multiple mass spectrometry steps can be combined in MS/MS to produce methods known in the art such as MS/MS/TOF, MALDI/MS/MS/TOF, and SELDI/MS/MS/TOF mass spectrometry.

The two primary methods for ionization of whole proteins are electrospray ionization (ESI) and matrix-assisted laser desorption/ionization (MALDI). The term “ionization” refers to the process of generating an analyte ion having a net electrical charge equal to one or more charge units. The term desorption refers to the removal of an analyte from a surface and/or the entry of an analyte into a gaseous phase. A particular form of desorption known as field desorption refers to methods in which a non-volatile test sample is placed on an ionization surface and an intense electric field is used to generate analyte ions.

The term “charge unit” refers in the usual sense to the fundamental electrical charge of a proton. Negative ions are those ions having a net negative charge of one or more charge units, while positive ions are those ions having a net positive charge of one or more charge units. The term “operating in negative ion mode” refers to those mass spectrometry methods where negative ions are detected. Similarly, “operating in positive ion mode” refers to those mass spectrometry methods where positive ions are detected.

Ions can be produced using a variety of methods including, but not limited to, electron ionization, chemical ionization, electrospray ionization, fast atom bombardment, field desorption, and matrix-assisted laser desorption ionization (“MALDI”), surface enhanced laser desorption ionization (“SELDI”), photon ionization, electrospray ionization, and inductively coupled plasma. In electron ionization, an analyte of interest in a gaseous or vapor phase interacts with a flow of electrons. Impact of the electrons with the analyte produces analyte ions, which may then be subjected to a mass spectroscopy technique. In chemical ionization, a reagent gas (e.g., ammonia) is subjected to electron impact and analyte ions are formed by the interaction of reagent gas ions and analyte molecules.

Another MS ionization technique is fast atom bombardment, in which a beam of high energy atoms (often Xe or Ar) impacts a non-volatile test sample, desorbing and ionizing molecules contained in the sample. Samples are dissolved in a viscous liquid matrix, such as glycerol, thioglycerol, m-nitrobenzyl alcohol, 18-crown-6 crown ether, 2-nitrophenyloctyl ether, sulfolane, diethanolamine, or triethanolamine. The choice of an appropriate matrix for a compound or sample is an empirical process.

In matrix-assisted laser desorption ionization, or “MALDI”, a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules. Matrix-assisted laser desorption ionization coupled with time-of-flight analyzers (“MALDI-TOF”) permits the analysis of analytes at femtomole levels in very short ion pulses.

In surface enhanced laser desorption ionization, or “SELDI”, a nonvolatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

Electrospray ionization, or “ESI”, methods pass a solution along a short length of capillary tube, to the end of which is applied a high positive or negative electric potential. Solution reaching the end of the tube is vaporized (e.g., nebulized) into a jet or spray of very small droplets of solution in solvent vapor. This mist of droplets flows through an evaporation chamber which is heated slightly to prevent condensation and to evaporate solvent. As the droplets get smaller, the electrical surface charge density increases until such time that the natural repulsion between like charges causes ions as well as neutral molecules to be released.

The method of atmospheric pressure chemical ionization, or “APCI”, is similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N2 gas may be used to improve removal of solvent. The gas-phase ionization in APCI can be more effective than ESI for analyzing less-polar species.

Inductively coupled plasma, or “ICP”, methods interact a sample with a partially ionized gas at a sufficiently high temperature to atomize and ionize most elements.

In those embodiments, such as MS/MS, where parent ions are isolated for further fragmentation, collision-induced dissociation (“CID”) is often used to generate the ion fragments for further detection. In CID, parent ions gain internal energy through collisions with an inert gas and subsequently fragment by a process referred to as “unimolecular decomposition”. Sufficient energy must be deposited in the parent ion so that certain bonds within the ion can be broken due to increased vibrational energy. Electron transfer dissciation (ETD) and high-collision energy dissociation (HCD) are two alternative fragmentation methods.

Proteases Generally

Proteases are often used to break proteins (e.g., from a proteome) into smaller fragments for analysis. Proteases (also known as proteinases or proteolytic enzymes), are a large group of enzymes that belong to the general enzyme class of hydrolases (e.g., they catalyze the hydrolysis of a chemical bond with the participation of a water molecule). Proteases occur naturally in all organisms and are involved in physiological reactions such as the digestion of food proteins to acting in highly regulated cascades (e.g., the blood-clotting cascade, the complement system, apoptosis pathways, and the invertebrate prophenoloxidase-activating cascade). Some proteases break specific peptide bonds (limited proteolysis) at particular amino acid sequences within a protein and some break down a complete peptide into its component amino acids (unlimited proteolysis). Activities and recognition sites of various proteases can be found, e.g., in MEROPS, a peptidase database (Rawlings, et al. MEROPS: the peptidase database. Nucleic Acids Res 2010, 38: D227-D233), available online at http://merops.sanger.ac.uk and in the Proteolysis Map CutDB database, http://www.proteolysis.org/proteases.

Proteases are involved in digesting long protein chains into short fragments, splitting the peptide bonds that link amino acid residues. Some of them can detach the terminal amino acids from the protein chain (exopeptidases, such as aminopeptidases, carboxypeptidase A); the others attack internal peptide bonds of a protein (endopeptidases, such as trypsin, chymotrypsin, pepsin, papain, elastase).

Proteases are divided into four major groups according to the character of their catalytic active site and conditions of action: serine proteinases, cysteine (thiol) proteinases, aspartic proteinases, and metalloproteinases. Attachment of a protease to a certain group depends on the structure of catalytic site and the amino acid (as one of the constituents) essential for its activity.

Proteases are used throughout an organism for various metabolic processes. Acid proteases secreted into the stomach (e.g., pepsin) and serine proteases present in duodenum (e.g., trypsin and chymotrypsin) enable organisms to digest the protein in food; proteases present in blood serum (e.g., thrombin, plasmin, Hageman factor) play an important role in blood-clotting, subsequent lysis of the clots, and the correct action of the immune system. Other proteases are present in leukocytes (e.g., elastase, cathepsin G) and play several different roles in metabolic control. Proteases determine the lifetime of other proteins that have important physiological roles, such as hormones, antibodies, or other enzymes. By complex cooperative action the proteases may proceed as cascade reactions, which result in rapid and efficient amplification of an organism's response to a physiological signal.

OmpT Protease

OmpT has an improved (e.g., narrower) substrate specificity relative to other proteases (e.g., trypsin). In particular, OmpT primarily cleaves between dibasic sites, rather than at single basic sites as does trypsin (e.g., after a single lysine or after a single arginine) (Dekker, N., et al. Substrate specificity of the integral membrane protease OmpT determined by spatially addressed peptide libraries. Biochemistry 40, 1694-1701 (2001); McCarter, J. D. et al. Substrate specificity of the Escherichia coli outer membrane protease OmpT. J Bacteriol 186, 5919-5925 (2004); Keijiro Sugimura, T. N. Purification, Characterization, and Primary Structure of Escherichia coli Protease VII with Specificity for Paired Basic Residues: Identity of Protease VII and OmpT. Journal of Bacteriology 170, 5625-5632 (1988); Sugimura, K. & Higashi, N. A novel outer-membrane-associated protease in Escherichia coli. J Bacteriol 170, 3650-3654 (1988)). The P1 position of the OmpT recognition sites are almost exclusively lysine or arginine. Studies suggest that, in addition to lysine and arginine residues, several other amino acid residues (e.g., alanine) are also allowed in its P1′ position in some instances, especially under denaturing conditions (Okuno, K. et al. Substrate specificity at the P1′ site of Escherichia coli OmpT under denaturing conditions. Biosci Biotechnol Biochem 66, 127-134 (2002)). Regardless of this range of amino acids found in the P1 and P1′ sites of the OmpT recognition site, the overall substrate specificity of OmpT is more stringent than trypsin.

In addition, OmpT has an efficient proteolytic activity and has an improved proteolytic activity in highly denaturing conditions relative to conventional proteases. The highest reported k_{cat}/Kₘ of OmpT is 1×10⁸ s⁻¹M⁻¹ when a fluorogenic tetrapeptide, e.g., Abz-Ala-Arg-Arg-Ala-Tyr(NO₂)—NH2 (Abz, o-aminobenzoyl; Tyr(NO₂), 3-nitrotyrosine) (SEQ ID NO: 1), was used as the substrate (Kramer, R. A., et al. In vitro folding, purification and characterization of Escherichia coli outer membrane protease ompT. Eur J Biochem 267, 885-893 (2000)). The catalytic efficiency of OmpT is substrate-dependent. Furthermore, OmpT is active in denaturing conditions. For some applications, Denaturants are required to expose buried OmpT cleavage sites in protein substrates to the enzyme for complete digestion. Owing to its rigid 10-stranded antiparallel beta-barrel structure, OmpT completely degrades recombinant proteins even in the presence of 4 M urea (White, C. B., et al. A novel activity of OmpT. Proteolysis under extreme denaturing conditions. J Biol Chem 270, 12990-12994 (1995)). Similarly, OmpT is compatible with detergents. OmpT is a membrane protein and, in some embodiments, is used with a detergent to remain soluble and maintain its active structure. OmpT has been shown to be compatible with zwitterionic, nonionic, and anionic detergents (Kramer, R. A., et al. In vitro folding, purification and characterization of Escherichia coli outer membrane protease ompT. Eur J Biochem 267, 885-893 (2000)). OmpT has an optimal activity at a pH close to neutral, e.g., 6.0-6.5 (Keijiro Sugimura, T. N. Purification, Characterization, and Primary Structure of Escherichia coli Protease VII with Specificity for Paired Basic Residues: Identity of Protease VII and OmpT. Journal of Bacteriology 170, 5625-5632 (1988); Kramer, R. A., et al. In vitro folding, purification and characterization of Escherichia coli outer membrane protease ompT. Eur J Biochem 267, 885-893 (2000)). Extreme (e.g., not close to neutral) pH conditions may bias digestion against basic or acidic protein substrates.

Active OmpT enzyme can be obtained through expression in the form of inclusion bodies and in vitro refolding (Kramer, R. A., et al. In vitro folding, purification and characterization of Escherichia coli outer membrane protease ompT. Eur J Biochem 267, 885-893 (2000)). The active enzyme can reach very high purity after a one-step purification. In some embodiments, The OmpT protease is liganded to LPS (lipopolysaccharide).

Database Searches

Proteomics relies on database search engines to interpret experimental mass spectral data. There are many available proteome database search algorithms for MS data interpretation, such as MASCOT, SEQUEST, DBDigger, Sonar, ProteinProspector, ProSite, and OMSSA. U.S. Pat. No. 6,940,065, the entire contents of which are incorporated herein by reference, describes a search process that can be used for mass spectra including a discussion of MASCOT and other search routines. Database search algorithms rely on a comparison between the theoretical fragmentation patterns of the database derived peptides and the experimentally observed fragmentation pattern. These search algorithms select a list of candidate database peptides, producing theoretical fragmentation patterns for each of them, and compare the theoretical spectrum to an experimentally measured MS spectrum. The theoretical peptide whose spectrum displays the highest spectrum similarity to the experimental spectrum is accepted as the best candidate and can be reported as identification.

Although the disclosure herein refers to certain illustrated embodiments, it is to be understood that these embodiments are presented by way of example and not by way of limitation.

### EXAMPLES

For the purposes of promoting an understanding of the principles of the technology, experiments were conducted wherein embodiments of the technology were demonstrated and reduced to practice.

Methods

Reagents

DNA restriction enzymes were purchased from Invitrogen and T4 DNA ligase was purchased from New England Biolabs. The pET28a vector and E. coli BL21(DE3) cells were obtained from EMD Biosciences. The SP-Sepharose media and the K16/20 cation exchange column were bought from GE Healthcare Life Sciences. Isopropyl-β-D-thiogalactopyranoside (IPTG) was from Roche; all other chemicals were purchased from either Thermo Fisher Scientific or Sigma-Aldrich unless otherwise noted. The fluorogenic substrate Abz-Ala-Arg-Arg-Ala-Tyr(NO₂)—NH₂ (Abz, o-aminobenzoyl; Tyr(NO₂), 3-nitrotyrosine) (SEQ ID NO: 1) was synthesized by the Protein Sciences Facility at the University of Illinois.

Cloning of the OmpT Gene and Construction of Expression Plasmid.

All PCR used Phusion Hot Start Polymerase (Finnzymes) and PCR-grade dNTPs (Invitrogen). PCR products and restriction-digested DNA were purified with the Qiaquick gel extraction and PCR cleanup kits (Qiagen). The OmpT gene was amplified from the genomic DNA of E. coli K12 DH5α. The primer sequences used for cloning OmpT were:

(SEQ ID NO: 2)
5′-ATGCGGGCGAAACTTCTGGGAATAG-3′
(forward) and
(SEQ ID NO: 3)
5′-TTAAAATGTGTACTTAAGACCAGCAGTAGTG-3′
(reverse).

Primers were synthesized by IDT. After the OmpT gene was cloned, another pair of primers containing restriction sites was used to amplify the gene without the N-terminal signal peptide with the sequences:

(SEQ ID NO: 4)
5′-ATTAATCCATGGCTTCTCGAGACTTTATCGTTTA-3′
and
(SEQ ID NO: 5)
5′-ACTCGGGAATTCTTAAAAGTGTACTTAAGACCAG-3′.

The amplified OmpT gene contains an NcoI restriction site at the 5′ end and an EcoRI site at the 3′ end (underlined). Both the pET28a vector and OmpT were doubly digested with NcoI and EcoRI (Invitrogen) and ligated to produce pNK1009, which was used to transform E. coli BL21(DE3) for protein expression after sequence confirmation by the University of Illinois Core DNA Sequencing Facility.

Protease Expression and Purification.

OmpT was expressed in inclusion bodies in BL21(DE3) as previously described with some modifications (Kramer, R. A., et al. In vitro folding, purification and characterization of Escherichia coli outer membrane protease ompT. Eur J Biochem 267, 885-893 (2000); Dekker, N., et al. In vitro folding of Escherichia coli outer-membrane phospholipase A. Eur J Biochem 232, 214-219 (1995)). Briefly, BL21(DE3) cells containing pNK1009 were grown overnight in 5 mL S.O.C. medium (20 g Bacto-Tryptone, 5 g Bacto Yeast Extract, 0.5 g NaCl, 2.5 mL of 1 M KCl, 20 mL of 1 M glucose in 1 L H₂O) with 50 mg/L kanamycin at 37° C. The 5 mL starter culture was inoculated into 1 L of S.O.C medium with 50 mg/L kanamycin and grown until the absorbance at 600 nm was between 1.0 and 1.5. The expression of OmpT inclusion bodies was induced by the addition of 1 M IPTG to a final concentration of 0.4 mM, followed by further incubation at 37° C. for 6-9 hours.

For OmpT purification, inclusion bodies were first isolated from the cell pellet as described with some modifications (Dekker, N., et al. In vitro folding of Escherichia coli outer-membrane phospholipase A. Eur J Biochem 232, 214-219 (1995)). Briefly, the cell pellet from a 1 L culture was resuspended in 12 mL lysis buffer (50 mM Tris-HCl, 40 mM EDTA, pH 8.0) and incubated with 3 mg lysozyme on ice for 30 min, and another 12 mL of pre-chilled lysis buffer was added quickly to introduce osmotic shock, followed by incubation on ice for another 30 minutes. The lysate was sonicated at 25 watts with a Sonic Dismembrator (Model 100, Fisher Scientific) every other minute until the lysate was no longer viscous. Inclusion bodies were collected by centrifugation at 4,500{g for 30 minutes. The pellet was washed once with 30 mL of wash buffer (10 mM Tris-HCl, 1 mM EDTA, pH 8.0) and extracted with 4 mL of dissolving buffer (8 M urea, 50 mM glycine, pH 8.3) on ice for 30 minutes.

To this solution, 16 mL of pre-chilled 31.25 mM N-dodecyl-N,N-dimethyl-3-ammonio-1-propanesulfonate (DodMe₂NPrSO₃) was added to initiate OmpT refolding. After 30 minutes on ice, the pH of the refolding mixture was adjusted to 4.0 using 10% acetic acid. The solution was centrifuged at 20,450×g, filtered and the supernatant loaded onto a 10 mL Fast Flow SP-Sepharose column (16 mm in diameter, 5 cm in length) equilibrated with buffer A (10 mM DodMe₂NPrSO₃, 20 mM sodium acetate, pH 4.0). The column was washed with 5 column volumes of buffer A and proteins were eluted from the column with a linear gradient of NaCl up to 2 M in 140-300 mL of buffer A. After cation exchange, OmpT was activated with lipopolysaccharide (LPS) (see Vandeputte-Rutten, L. et al. Crystal structure of the outer membrane protease OmpT from Escherichia coli suggests a novel catalytic site. EMBO J 20, 5033-5039 (2001); Kramer, R. A. et al. Lipopolysaccharide regions involved in the activation of Escherichia coli outer membrane protease OmpT. Eur J Biochem 269, 1746-1752 (2002)) and dialyzed against enzymatic buffer to remove high concentration salt, after which LPS-bound OmpT was found in two forms due to a single self-degradation site (R217-K218) (Kramer, R. A., et al. In vitro folding, purification and characterization of Escherichia coli outer membrane protease ompT. Eur J Biochem 267, 885-893 (2000)). Greater than 80% of the enzyme was isolated in its intact form. Based on SDS-PAGE analysis, fractions containing OmpT were pooled, aliquoted, and frozen at −80° C. for storage after the OmpT activity was confirmed using the synthetic fluorogenic substrate Abz-Ala-Arg-Arg-Ala-Tyr(NO₂)—NH₂ (SEQ ID NO: 1) (Kramer, R. A., et al. In vitro folding, purification and characterization of Escherichia coli outer membrane protease ompT. Eur J Biochem 267, 885-893 (2000)).

Preparation of Standard Proteins and High-Mass Proteome Samples.

The standard proteins carbonic anhydrase, glyceraldehyde 3-phosphate dehydrogenase (GAPDH), and phosphorylase b were directly dissolved in 8 M urea to make 2-5 mg/mL stock solutions. Bovine serum albumin (BSA) was reduced in 5 mM dithiothreitol (DTT), alkylated with 10 mM iodoacetamide in the dark, and precipitated with ice-cold acetone before resuspension in 8 M urea for OmpT digestion. For the human proteome sample, HeLa S3 cells were obtained from the American Type Culture Collection and grown as previously described (Lee, J. E. et al. A robust two-dimensional separation for top-down tandem mass spectrometry of the low-mass proteome. J Am Soc Mass Spectrom 20, 2183-2191 (2009)). Cells were lysed by boiling in cell lysis buffer (4% SDS, 100 mM Tris-HCl1, 10 mM DTT, pH 7.5) for 10 minutes, incubated with 100 mM iodoacetamide for 30 min in the dark, aliquoted, and frozen at −80° C. for future use. To fractionate the whole proteome into a ladder of molecular mass bins, a continuous tube-gel electrophoresis technology, Gel-eluted Liquid Fraction Entrapment Electrophoresis (GELFrEE), was applied for primary separation (Tran, J. C. & Doucette, A. A. Multiplexed size separation of intact proteins in solution phase for mass spectrometry. Anal Chem 81, 6201-6209 (2009)). Specifically, an eight-channel, multiplexed commercial continuous tube-gel electrophoresis device (GELFREE 8100 fractionation system, Protein Discovery Inc.) was used with 8% or 10% gel cartridges (Protein Discovery) to prepare the high-mass HeLa proteome. The HEPES-SDS buffer system, pH 7.8, was used as recommended by the vendor. To load samples onto the GELFrEE devices, protein concentrations were measured using the BCA assay and aliquoted HeLa lysates corresponding to 1-2 mg of total protein were thawed on ice, precipitated by cold acetone at −20° C. for 30 minutes, air-dried before resuspension with sample loading buffer, and then heated at 50° C. for the commercial GELFrEE. After sample loading, the commercial GELFrEE device was operated as described in the manufacturer's instructions. Each fraction contained 1.2 mL of sample volume (150 μL for each channel; samples from eight channels were pooled together for the same fraction) and fractions corresponding to the high-mass proteome (˜20-100 kDa) were cleaned by cold acetone precipitation and air-dried prior to resuspension in 8 M urea for OmpT digestion.

OmpT Digestion and Sample Clean-Up.

To obtain active enzyme, aliquoted OmpT solution was thawed on ice, activated with 0.1 mM LPS overnight (Vandeputte-Rutten, L. et al. Crystal structure of the outer membrane protease OmpT from Escherichia coli suggests a novel catalytic site. EMBO J 20, 5033-5039 (2001); Kramer, R. A. et al. Lipopolysaccharide regions involved in the activation of Escherichia coli outer membrane protease OmpT. Eur J Biochem 269, 1746-1752 (2002)), and dialyzed against enzymatic buffer (10 mM Bis-Tris-HCl, 2 mM EDTA, pH 6.0). Immediately after dialysis, OmpT (liganded to LPS) was mixed with resuspended standard proteins or high-mass HeLa GELFrEE samples and incubated at 22° C. overnight. Digested standard proteins or GELFrEE samples were cleaned up by methanol-chloroform precipitation (Lee, J. E. et al. A robust two-dimensional separation for top-down tandem mass spectrometry of the low-mass proteome. J Am Soc Mass Spectrom 20, 2183-2191 (2009); Wessel, D. & Flugge, U. I. A method for the quantitative recovery of protein in dilute solution in the presence of detergents and lipids. Anal Biochem 138, 141-143 (1984)) before solubilizing at 100° C. in sample loading buffer and were loaded onto a single-channel custom GELFrEE device for secondary continuous tube-gel electrophoresis separation (Tran, J. C. & Doucette, A. A. Gel-eluted liquid fraction entrapment electrophoresis: an electrophoretic method for broad molecular weight range proteome separation. Anal Chem 80, 1568-1573 (2008)). The buffer system of this custom device was Tris-glycine (25 mM Tris, 0.2 M glycine, 0.1% SDS). Tube gels with Tris-glycine were cast at 15% T in this secondary continuous tube-gel electrophoresis for resolving digested peptides. The custom GELFrEE device was operated at 180 V and 16 fractions were collected containing proteins up to 30 kDa over 100 minutes. SDS was removed from collected fractions by methanol-chloroform precipitation. The resultant protein pellets from either standard protein digestions or GELFrEE digestions by OmpT were recovered by buffer A (95% H₂O, 5% acetonitrile, 0.2% formic acid) solubilization and injected onto a nanoLC coupled to a mass spectrometer for on-line characterization as described below.

Nanocapillary Liquid Chromatography-Mass Spectrometry (nanoLC-MS/MS).

A PLRP-S trap column (New Objective, Inc.), 150 μm inner diameter (i.d.) with a 3 cm media length, was used for sample loading, followed by a 10 cm long×75 μm i.d. PLRP-S analytical column for sample separation. A linear gradient flowing at 300 nL/minute from an Eksigent 2D system started from 95% buffer A and 5% buffer B (5% H₂O, 95% acetonitrile, 0.2% formic acid), ramped to 40% B in 55 minutes, and finally 85% B in 15 minutes. Samples eluted from the nanoLC were electrosprayed into a custom hybrid linear ion trap Fourier transform ion cyclotron resonance mass spectrometer (11 Tesla LTQ-FT-Ultra mass spectrometer, Thermo Fisher Scientific). Samples were analyzed using a data-dependent top 2 or top 3 method. Collision-induced dissociation (CID) was applied with a 10-15 m/z isolation window and normalized collision energy of 41%; for MS1, 1-6 microscans at 160,000 resolving power at 400 m/z were used with a target value of 1 million and scan range of m/z 450-1800 in the Fourier transform ion cyclotron resonance cell (FT-ICR); for MS2, 2-6 microscans at 80,000 resolving power were used with a target value of 1-1.5 million in the FT-ICR. For CID and ETD comparison analysis, a Velos Orbitrap Elite system was used. Samples were analyzed either using a data-dependent top 3 or 5 method in separate CID or ETD runs, or top 2 or 3 method in alternating CID and ETD runs. Both CID and ETD were applied with a 15 m/z isolation window; normalized collision energy for CID was set at 41% and reaction time for ETD was 5-25 ms. For MS1, 2-4 microscans at 120,000 resolving power at 400 m/z were used with a target value of 1 million and scan range of m/z 400-1500 in orbitrap; for MS2, 3-6 microscans at 60,000 resolving power were used with a target value of 1 million in the orbitrap.

Data Reduction and Database Searching.

Each LC-MS/MS run was collected as a “.raw” file and processed with ProSightPC 2.0 SP1 software (Thermo Fisher Scientific). Briefly, monoisotopic neutral precursor and fragment masses were determined using the Xtract algorithm, complied into a “.puf” file (ProSight Upload Format), and searched on a 168-core cluster in two different search modes (absolute mass and biomarker) against two shotgun-annotated human proteome databases.

Biomarker search mode does not assume any hypothetical cleavages in the database and queries every possible sub-sequence of any protein in the intact protein database (UniProt release 2011-10) for a match within the defined mass tolerance window. In this mode, the precursor mass tolerance window was set to 1.1 Da and the fragment mass tolerance was set to ±10 ppm. To estimate the false discovery rate (FDR) in biomarker search mode, a q value evaluation approach was applied as previously described (Tran, J. C. et al. Mapping intact protein isoforms in discovery mode using top-down proteomics. Nature 480, 254-258 (2011)). A decoy database was built by scrambling the protein sequences from the forward intact database (Elias, J. E. & Gygi, S. P. Target-decoy search strategy for increased confidence in large-scale protein identifications by mass spectrometry. Nat Methods 4, 207-214 (2007)). All data were searched against both the forward and decoy databases separately using identical search parameters. All search hits were scored using a Poisson-based model (p score) (Meng, F. et al. Informatics and multiplexing of intact protein identification in bacteria and the archaea. Nat Biotechnol 19, 952-957 (2001)) and a posterior probability-based q value was calculated for each hit to estimate the FDR for each identification event (Benjamini, Y. & Hochberg, Y. Controlling the false discovery rate—a practical and powerful approach to mulpiple testing. J. R. Stat. Soc. Ser. B-Methodol. 57, 289-300 (1995); Storey, J. D. & Tibshirani, R. Statistical significance for genomewide studies. Proc Natl Acad Sci USA 100, 9440-9445 (2003)).

For the absolute mass search, a custom peptide database was constructed using the OmpT cleavage propensities (P1=(K, R); P1′=(K, R, A, S, G, V, I, L)) determined by biomarker search hits. Eight missed cleavages were considered in constructing this Middle Down database, which contained 20 million peptide forms (including signal peptides, alternative splice variants, and PTMs). To search data in absolute mass mode, ProSightPC iterative searching was used, with the precursor mass tolerance window set to 2.2 Da and the fragment tolerance set to ±10 ppm for the first level search; an 81 Da precursor mass tolerance and ±10 ppm fragment tolerance were used for the second level search. FDR estimation was performed as described above.

Peptide hits with a q value lower than 0.01 (1% FDR cut-off) from both the biomarker and absolute mass search modes were reported and used for further analysis in this study. A brief comparison was drawn between biomarker hits and absolute mass hits. ProteinCenter software (Thermo Fisher Scientific) was used to group peptides and cluster protein identifications for unique protein counting

### Example 1

### Verification of OmpT Activity for the Production of Large Peptides Based on Computational Analysis

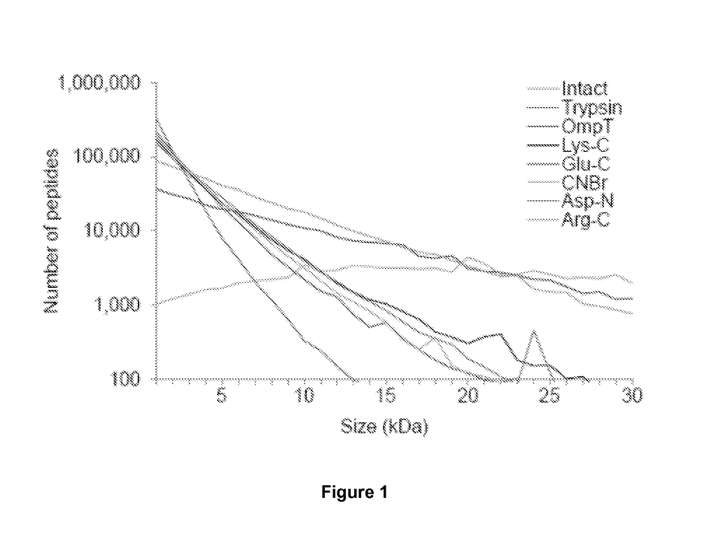

During the development of embodiments of the technology provided herein, data were collected to compare the activities of various proteases and select an enzyme with which to digest the human proteome into appropriately sized peptides. In particular, in silico digestions were performed using various enzymatic or chemical cleavage rules to visualize the differences of expected peptide abundances in different mass bins (FIG. 1). Most of the conventional enzymatic approaches (e.g., trypsin, Lys-C, Arg-C, Glu-C, Asp-N), especially trypsin, produce predominantly small peptides (<2 kDa), drastically increasing sample complexity after digestion. OmpT, which cleaves at less common dibasic sites, produced fewer small peptides. Traditional digestion methods generated very few peptides larger than 3 kDa while OmpT created many large peptides, even up to 30 kDa. To assess the results of potential missed cleavage sites, another in silico digestion was performed assuming 2 missed cleavages; predicted peptide size distributions were similar to the case in which there were no missed cleavages.

### Example 2

### OmpT Digestion Conditions

During the development of embodiments of the technology provided herein, experiments were performed to determine appropriate digestion conditions for OmpT. Four standard proteins (carbonic anhydrase, glyceraldehyde 3-phosphate dehydrogenase (GAPDH), bovine serum albumin (BSA), and phosphorylase b) were used as test substrates. The data collected demonstrated that OmpT is most efficient at digesting these substrates at pH 6.0 with 2-3 M urea present after overnight incubation at 22° C. Urea was used to reduce any higher-order structure present in the test substrates. Incubation at 22° C. was selected instead of 37° C. to avoid carbamylation adducts from urea. Surprisingly, OmpT was more active at 22° C. than at 37° C. under these conditions. No observable level of carbamylation (+43 Da) on lysines suggested that 22° C. incubation is in fact optimal for reducing side reactions while maximizing protease activity.

### Example 3

### OmpT Reactivity Toward Standard Proteins

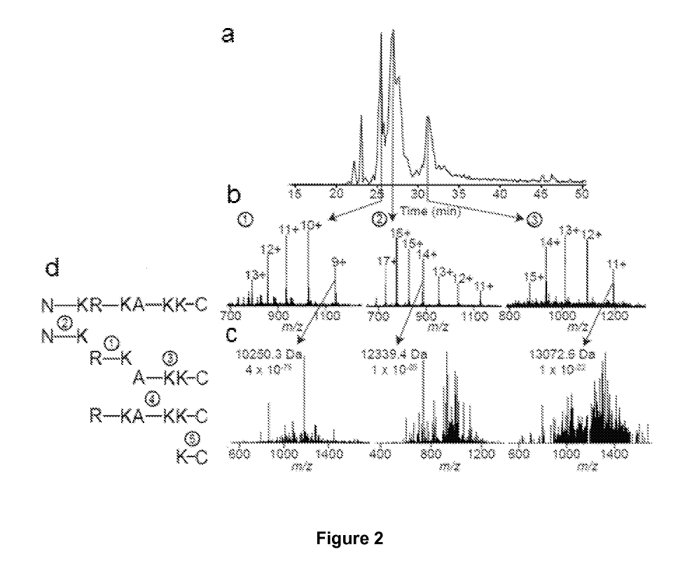

During the development of embodiments of the technology provided herein, data were collected from experiments testing the reactivity of OmpT using standard proteins as substrates. Using the digestion conditions determined above, test substrates were completely depleted (by visual inspection of SDS-PAGE gels) in 10 hours at a substrate:enzyme ratio of up to 75:1, with the substrate concentration at 0.3-0.75 mg/mL. As an example, peptide products from GAPDH digestion by OmpT were visualized on a Coomassie-stained SDS-PAGE gel, characterized via nanoLC-MS/MS (FIG. 2a-c), and identified by ProSightPC, with cleavage sites highlighted in the peptide map aligned with the original GAPDH sequence (FIG. 2d). In addition to predicted dibasic cleavages, a K-A cleavage was observed, demonstrating that OmpT cleaves under certain conditions at sites comprising other aliphatic amino acid residues at the P1′ position, e.g., especially under extreme denaturing conditions (see, e.g., Okuno, K. et al. Substrate specificity at the P1′ site of Escherichia coli OmpT under denaturing conditions. Biosci Biotechnol Biochem 66, 127-134 (2002)). In additional experiments, a K-A cleavage product was detected, e.g., both on a gel and by LC-MS/MS (FIG. 2d, peptide 4).

Although there is a K-K site within the GAPDH sequence, the cleaved product at this site (peptide 5 in FIG. 2d) was barely observable in the LC-MS/MS run. This is likely because the flanking amino acid residues in the P2 and P3 positions are both aspartic acid residues; these negative charges may prevent the binding of the nearby K-K site to the negatively charged active site of OmpT (see, e.g., Vandeputte-Rutten, L. et al. Crystal structure of the outer membrane protease OmpT from Escherichia coli suggests a novel catalytic site. EMBO J 20, 5033-5039 (2001)).

The other three standard protein digestions were also visualized on Coomassie-stained gels. Peptides from carbonic anhydrase, GAPDH, and phosphorylase b were illustrated along with their cleavage sites highlighted in the protein sequences. These experiments demonstrated 100% sequence coverage for both GAPDH and carbonic anhydrase, and demonstrated 84% coverage for phosphorylase b using the identified peptides. Although peptides from BSA resulting OmpT cleavages were readily seen on Coomassie-stained gels, no peptides were confidently identified, mostly likely due to their large sizes.

### Example 4

### Middle Down Proteomics Based on OmpT

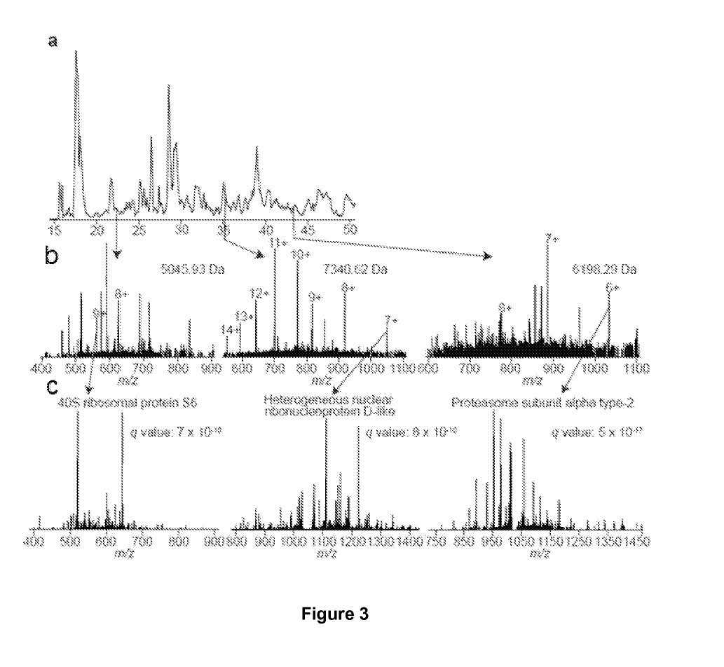

During the development of embodiments of the technology provided herein, an OmpT-based platform for Middle Down proteomic analysis was established to analyze complex proteome samples. Specifically, a human HeLa proteome sample was separated by multiplexed primary continuous tube-gel electrophoresis into fractions containing a distribution of protein sizes with the best resolution from 20 to 100 kDa. The fractionated samples in this mass region were precipitated with cold acetone, resuspended in 8 M urea and digested with OmpT at a ratio of 25:1 (final protein concentration of ˜0.5 mg/mL and 3 M urea). Digested samples underwent a secondary continuous tube-gel electrophoresis separation and methanol-chloroform precipitation prior to injection on nanoLC-MS/MS. In one representative example from the Middle Down pipeline (FIG. 3), 109 unique peptides with an average length of 6.4 kDa were identified from 67 unique proteins in a single run. From the entire Middle Down analysis on the high-mass HeLa proteome (20-100 kDa), 3697 unique peptides (average size: 6.3 kDa) from 1038 unique proteins (26% average sequence coverage) were identified at an estimated 1% false discovery rate (FDR). Among these peptides, 2493 were confidently identified with an intact peptide tolerance <10 ppm without manual verification; peptides with intact mass discrepancies outside this window were identified with multiple matching fragment ions <10 ppm but were not further pursued in this study. To eliminate the possibility that observed peptides may have come from auto-degradation during sample manipulation and not from OmpT digestion, a negative control was used in which all conditions were identical except the addition of OmpT. This control experiment led to very few confident identifications, indicating that the observed peptides were due to OmpT digestion of substrate proteins.

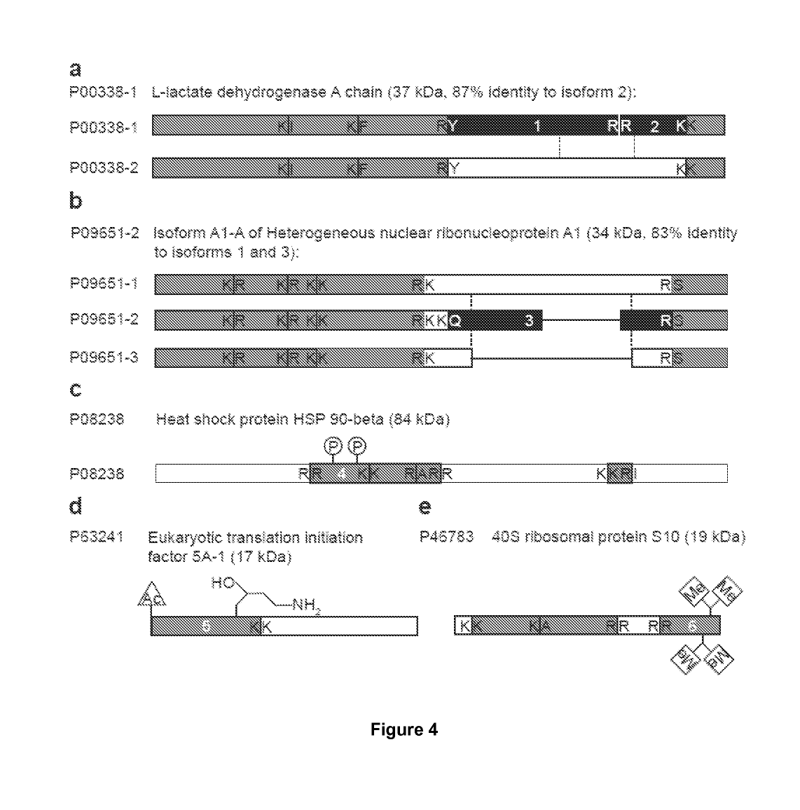

Furthermore, data were collected to differentiate specific protein isoforms based on proteotypic OmpT peptides (FIG. 4). Detailed sequence alignments between protein isoforms revealed areas of sequence identity, while OmpT peptides, owing to their desirably large size, covered those unique regions where isoform sequences differed. Longer peptides are also beneficial for PTM identification. In this study, ˜25% of OmpT peptides were identified with PTMs (using annotated modifications from the UniProt database) (Tran, J. C., et al. Mapping intact protein isoforms in discovery mode using top-down proteomics. Nature 480, 254-258 (2011); Lee, J. E. et al. A robust two-dimensional separation for top-down tandem mass spectrometry of the low-mass proteome. J Am Soc Mass Spectrom 20, 2183-2191 (2009)); examples of multiply modified peptides are shown (FIG. 4c-4e). Together, these data show that OmpT peptide-based analysis leads to more biologically informative findings. For example, these experiments demonstrate that peptides from OmpT digestion allow for the identification and characterization of protein isoforms and PTM combinations that may not be easily accessible by other protease-based proteomic approaches.

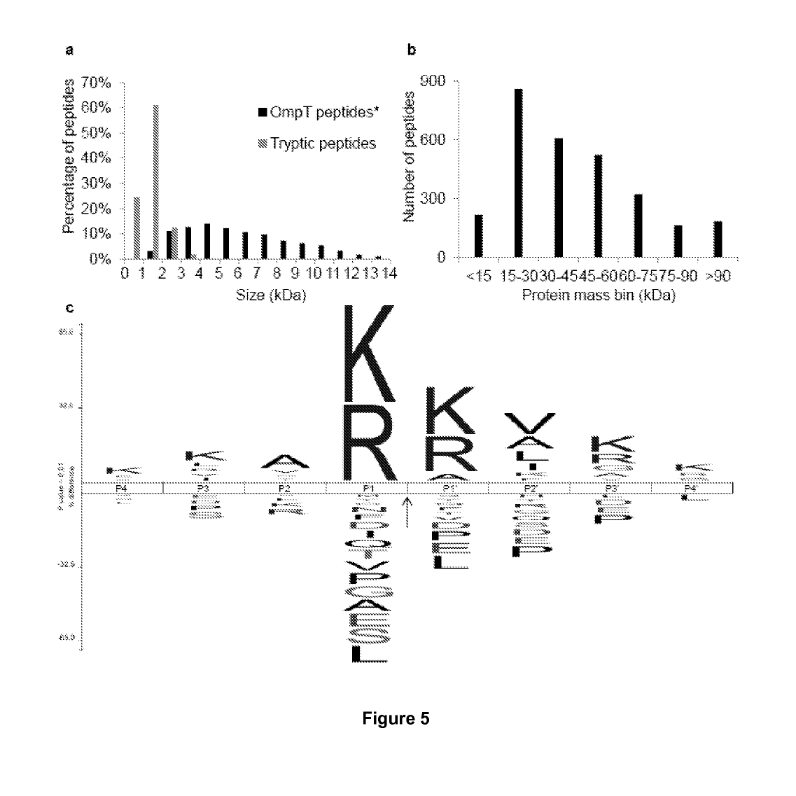

During the development of embodiment of the technology described, the mass distribution of identified OmpT peptides was profiled by plotting peptide mass frequencies in 1-kDa mass bins (up to 14 kDa to ease analysis) in comparison with tryptic peptides (FIG. 5a). Although the average size of identified OmpT peptides was 6.3 kDa, the actual average peptide size based on silver-stained gels is estimated to be higher than 6.3 kDa because many peptides having masses greater than 10 kDa are readily visible on gels. The OmpT-based Middle Down platform demonstrates its robustness across the proteome based on the identified peptide numbers (FIG. 5b) from different mass bins after primary continuous tube-gel electrophoresis. In some embodiments, by decreasing the crosslinking in the primary continuous tube-gel electrophoresis device, significantly better separations were achieved above 100 kDa, making this very high mass proteome accessible for OmpT digestion.

The entire dataset was searched using ProSightPC in “biomarker” mode against an intact protein database. A biomarker search assumes no proteolytic cleavage and queries every possible sub-sequence of each protein in the database. Biomarker peptide hits with a mass difference <10 ppm were then selected to extract the P4-P4′ recognition sites for the generation of an unbiased consensus sequence for OmpT. The observed amino acid frequency was normalized using a reference set of genomic amino acid frequencies to provide relative amino acid frequencies (P4-P4′) at the OmpT cleavage sites.

As shown in FIG. 5c, the P1 site allows almost exclusively lysine (51%) and arginine (42%) residues, while the P1′ site is more permissive, mainly allowing lysine (29%), arginine (23%), as well as alanine (11%) and serine (8%) residues. Some minor amino acid residues were also observed at the P1′ site. OmpT has a specificity at the P1′ site that includes lysine and arginine and other amino acids in a relatively minor proportion. Based on these data, the “major cleavage sites” are K/R-K/R/A/S. An in silico digest using these major cleavage sites in the proteome and allowing 0 and 2 missed cleavages produced a peptide size distribution that strongly resembles the distribution assuming only K/R-K/R cleavages.

Interestingly, in addition to selectivities at P1 and P1′ sites, the P2′ site has a mild preference for aliphatic amino acid residues such as valine, alanine, leucine and isoleucine over others. Furthermore, while OmpT favors positively charged residues across its recognition sites (with the exception of P2), it has an overall repulsion of negatively charged and proline residues. Selectivities outside P1-P1′ have been previously reported and might explain our observation that the actual average number of missed cleavages at the major sites is 0.99±1.29. In spite of these preferences, OmpT is still a stringent protease with well-defined substrate specificities, which will be better understood with future experimentation and data mining.

A brief performance comparison between collision induced dissociation (CID) and electron transfer dissociation (ETD) was made using OmpT peptides from three fractions of secondary continuous tube-gel electrophoresis. Technical replicates were analyzed in a single run with alternating CID and ETD on the same precursors, or in separate runs where only one fragmentation technique was used. While the former led to a 48% overlap in peptide identifications, ETD versus CID in separate runs only gave a 23% overlap. These results suggest that ETD and CID both serve as effective and highly complementary fragmentation approaches to identify and characterize OmpT peptides.

A brief performance comparison between collision induced dissociation (CID) and electron transfer dissociation (ETD) was made using OmpT peptides from three fractions of secondary continuous tube-gel electrophoresis. Technical replicates were analyzed in a single run with alternating CID and ETD on the same precursors or in separate runs where only one fragmentation technique was used. While the former led to a 48% overlap in peptide identifications, ETD versus CID in separate runs only gave a 23% overlap. These results suggest that ETD and CID both serve as effective and highly complementary fragmentation approaches to identify and characterize OmpT peptides.

All documents, publications, and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in biochemistry, molecular biology, proteomics, or related fields are intended to be within the scope of the following claims.

We claim:

1. A method for identifying a polypeptide, the method comprising:
a) contacting the polypeptide with an OmpT protease to produce a fragment; and
b) analyzing the fragment by mass spectrometry to generate a mass spectrum.

2. The method of claim 1 wherein the OmpT protease is isolated from Escherichia coli.

3. The method of claim 1 wherein the OmpT protease is cloned from Escherichia coli.

4. The method of claim 1 wherein the OmpT protease is a mutant OmpT protease.

5. The method of claim 1 wherein the fragment has a mass that is greater than 2 kDa.

6. The method of claim 1 wherein the fragment has a mass that is greater than 10 kDa.

7. The method of claim 1 wherein the fragment has a mass that is greater than 30 kDa.

8. The method of claim 1 wherein the contacting occurs in the presence of a denaturant.

9. The method of claim 1 wherein the contacting occurs in approximately 2-3 M urea, at approximately 22° C., and at about a pH of 6.

10. The method of claim 1 wherein the contacting occurs for approximately 8 to 24 hours and a ratio of the polypeptide to the OmpT protease that is approximately 10:1 to 200:1.

11. The method of claim 1 further comprising comparing the mass spectrum to a database.

12. The method of claim 1 wherein the fragment identifies an isoform of the polypeptide or a post-translational modification of the polypeptide.

13. The method of claim 1 further comprising purifying the polypeptide by continuous tube-gel electrophoresis.

14. A method for identifying a polypeptide, the method comprising:
a) contacting the polypeptide with a protease that specifically cleaves at a two-amino acid recognition site to produce a fragment; and
b) analyzing the fragment by mass spectrometry to generate a mass spectrum.

15. The method of claim 14 wherein the two-amino acid recognition site comprises a dibasic site

16. The method of claim 14 wherein the first amino acid of the two-amino acid recognition site is a lysine or an arginine and the second amino acid of the two-amino acid recognition site is a lysine or an arginine.

17. The method of claim 14 wherein the first amino acid of the two-amino acid recognition site is a lysine or an arginine and the second amino acid of the two-amino acid recognition site is an alanine.

18. The method of claim 14 wherein the first amino acid of the two-amino acid recognition site is a lysine or an arginine and the second amino acid of the two-amino acid recognition site is a lysine, an arginine, an alanine, a serine, a glycine, a valine, an isoleucine, or a leucine.

19. A kit comprising an OmpT protease, a buffer, a negative control polypeptide, and/or a positive control polypeptide.
